(19) United States
(12) Patent Application Publication
(10) Pub. No.: US 2013/0090699 A1
(43) Pub. Date: Apr. 11, 2013

(54) INSTRUMENTS FOR PRODUCING A REINFORCEMENT OF A HUMAN SPINAL COLUMN

(71) Applicant: Ulrich Holzwarth, (US)

(72) Inventor: Ulrich Holzwarth, (US)

(21) Appl. No.: 13/267,461

(22) Filed: Oct. 6, 2011

(51) Int. Cl. A61B 17/86; A61B 17/16

(52) U.S. Cl. 606/309; 606/79

(57) ABSTRACT

The invention relates to instruments for reinforcing a human spinal column, in particular in the region of the last vertebral body S1 of the sacrum toward the head and of the lowermost lumbar vertebral body L5 or additionally of the second lowest lumbar vertebral body L4, comprising a tissue protection cover to keep open a position on the sacrum, a tool for removing bone tissue from the vertebral body S1 in the case of single level supply and from the vertebral bodies S1 and L5 in the case of double level supply, at least one tool for removing intervertebral disc tissue between S1 and L5 and a reinforcement screw consisting of a pin with a first, leading, proximal threaded pin portion and a second, driving, distal threaded pin portion.

### FIELD OF THE INVENTION

The invention relates to instruments for producing a reinforcement of a human spinal column, in particular of the region of the last vertebral body S1 of the sacrum toward the head and of the lowermost lumbar vertebral body L5 or additionally of the second lowest lumbar vertebral body L4, comprising a tissue protection cover to keep open an access on the sacrum, a tool for the secured removal of bone tissue from vertebral bodies in the sacrum and at least one tool for removing intervertebral disc tissue between S1 and L5 and optionally between L4 and L5 and a reinforcement screw with at least a first, leading, proximal threaded pin portion and a second, driving, distal threaded pin portion.

### BACKGROUND OF THE INVENTION

Implant screws of this type are called Herbert screws. With regard to the Herbert screw, reference is made, for example, to DE-A-2 807 364. The screw is shown here in particular as a surgical fastening means in order to join bone fracture parts together by means of contraction and to thus accelerate healing with a certain compression of the fracture parts.

In the published application mentioned, however, the Herbert screw is also described for use to implement a distraction between two bones or fracture elements. In these interventions, the two bones or fracture parts are initially pre-drilled with different diameters before the so-called Herbert screw is inserted. In order to achieve a screwing operation which is as simple and targeted as possible, the Herbert screw is already provided with a central through-cannulation, through which a centering pin or guide wire can be guided.

Further examples of osteosynthetic compression screws are furthermore shown in WO-A-9109572, in which the intermediate portion tapers conically, or FR-A-2808182, in which the tip of the distal portion is designed as a drill bit. Lastly, WO-A-2004/049915 also shows a version of a Herbert screw, which has a central injection canal and has outlet openings in the intermediate portion, through which a liquid can be ejected into this region. The screw mentioned last here is used, in particular, for surgical interventions in patients with a prolapse or slipped disc.

As life goes on, regressive changes occur within the intervertebral disc tissue and these are accompanied by a reduction in the water content and therefore also in the turgor. The consequence of this is that the intervertebral discs become thinner. This leads to a loss in stability in the movement segment, while with age the resistance of the annulus fibrosus, namely the cover of the intervertebral disc, decreases. Thus, the tissue of the gelatinous core (nucleus pulposus) inevitably gives way in the direction of the weak points under load. Intervertebral disc protrusions firstly occur. If the fibrous ring of the annulus completely tears, the prolapse of the lumbar intervertebral disc is produced owing to the escaping gelatinous core. In the process, the content of the intervertebral foramen, namely the nerve roots and accompanying vessels, may be compressed. This is treated by surgery in the case of prolapse of the intervertebral disc by distracting Herbert screws. In this case, drilling takes place from the sacrum through the following lumbar vertebra to that region where the lumbar prolapse of the intervertebral disc occurred. The implant screw is set, a guide wire optionally being introduced through the central cannulation in order to insert the screw as precisely as possible in the direction aligning with the bore. This is also not guaranteed despite a guide wire.

EP-B-1257217 discloses a device which is to allow access to a series of adjacent vertebral bodies within the human spinal column, said device having a tool to form a bore and an axial implant. The implant that can be implanted in the transsacral axial bore extends toward the head through at least one vertebral body toward the foot and in or through at least one vertebral body toward the head, the relevant vertebral bodies having been drilled through beforehand.

Furthermore, the device has an anterior tract cover for handling the tool and implant and for exposing an anterior sacral position of an anterior sacral vertebral body or a posterior sacral position of a posterior sacral vertebral body, the use of a tract cover to protect soft part tissue being obvious and indispensable at least in an anterior position. Accordingly, the aforementioned drilling tool is arranged in the tract cover to protect the surrounding tissue. The tract cover is not used for the drilling itself. The bone tissue of the vertebral bodies is mechanically and thermally destroyed when drilled through and is therefore lost. In addition, the vertebral bodies are weakened.

The means mentioned are separate units.

The vertebral bodies have a relatively large fraction of spongy bone material. Threads with relatively high flanks are required for anchoring an implant screw in this region. There are, however, limits to this as, otherwise, a risk of injury is increased. An improved anchoring of the implant screw in the spongy bone material cannot therefore be achieved with obvious means, in other words with an elevation of the thread flanks alone.

### SUMMARY OF THE INVENTION

It is therefore an object of the present invention to develop instruments for reinforcing a human spinal column in order to, on the one hand, reduce the surgical outlay, to obtain bone tissue removed in the process and to obtain it in a reusable manner and, on the other hand, to improve the anchoring of implants in spongy bone material.

This object is achieved by instruments for reinforcing a human spinal column, in particular in the region of the last vertebral body S1 of the sacrum toward the head and of the lowermost lumber vertebral body L5 or additionally also of the second lowest lumber vertebral body L4, comprising a tissue protection for keeping open a position on the sacrum, a tool for the secured removal of bone tissue from vertebral bodies, optionally with an adjustable working depth, optionally a Kirschner wire or the like guided in or along the tissue protection, at least one tool for removing intervertebral disc tissue between S1 and L5 and optionally between L4 and L5, for example by means of a surgical instrument according to DE-U-202008016969, as well as a reinforcement screw with at least a first, leading, proximal (i.e. close to the body, viewed from the patient) threaded pin portion and a second, driving distal (remote from the body) threaded portion, at least the proximal, leading threaded portion being provided with a self-cutting thread. The self-cutting thread of a wooden screw penetrates directly into the bone tissue and compresses the latter by displacement while avoiding a preceding drilling.

By the punching out of a bone plug, the bone tissue is retained undestroyed and can be used to fill the intermediate space between two vertebral bodies. The removed tissue is comminuted and optionally mixed with bone replacement material. Tissue-destroying drilling is therefore not necessary. An initial or prior drilling is also not necessary.

The cross-section of the bone plug and internal cross-section of the punching tool is polygonal and smaller than the core diameter of the reinforcement screw (in the proximal threaded portion) and therefore allows a firmer hold of the bone screw, in turn with simultaneous compression of the spongy tissue.

In single level supply (S1-L5), no tissue is punched out in L5 (with a reinforcement screw with two portions) or in double level supply (S1-L5-L4) no tissue is punched out in L4 (with a reinforcement screw with three portions). The proximal part of the reinforcement screw is introduced in a self-cutting/self-drilling manner. In the uppermost lumber vertebral body to be supplied toward the head in each case, tissue removal is deliberately dispensed with in order to obtain tissue compression by means of the self-cutting thread and therefore a better seat of the reinforcement screw.

Advantageous configurations of the subject of the invention and the significance and mode of action thereof will be described in the following description with reference to the associated drawings. A preferred embodiment of the subject of the invention is shown in the drawings and described in more detail below.

### BRIEF DESCRIPTION OF THE DRAWINGS

FIG. 1 shows a side view of a preferred embodiment of the reinforcement screw with two portions and

FIG. 2 shows a further embodiment of the reinforcement screw with three portions and

FIG. 3 shows a side view of a tool for the secured removal of bone tissue from vertebral bodies in a schematic view.

### DESCRIPTION OF THE PREFERRED EMBODIMENT

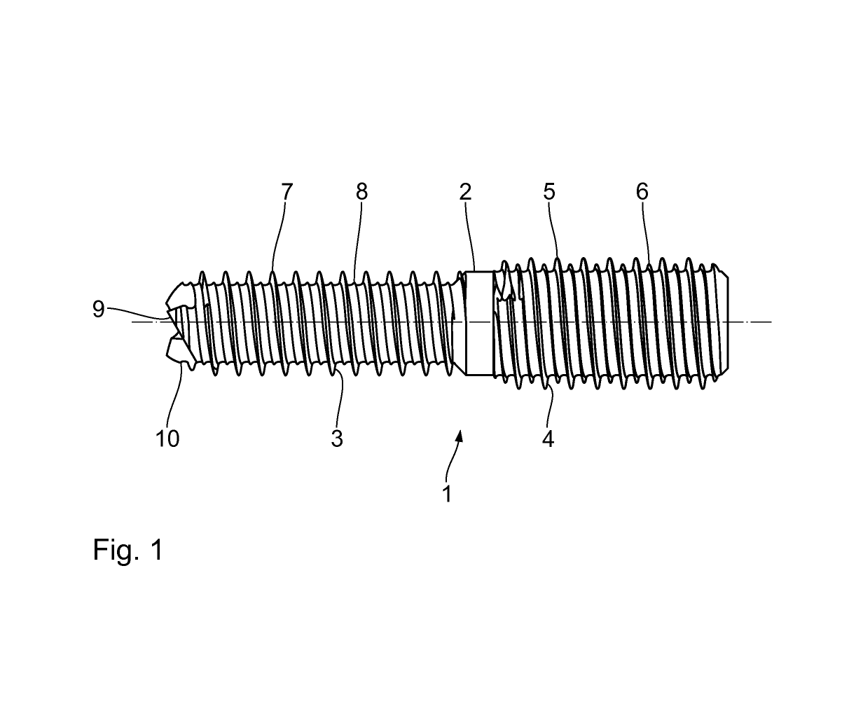

FIG. 1 shows the reinforcement screw in its entirety in a side view and designated 1. It is produced from a pin 2. This generally consists of titanium or a body-compatible permitted titanium alloy. The pin 2 has a first proximal (close to the body) threaded portion 3, which is head-free, and a second, distal threaded portion 4. The first threaded portion 3, when introduced into the patient, lies to the front close to the body and is therefore also called the leading threaded portion. Viewed from the patient, the leading threaded portion is closer to the body and is designated the proximal threaded portion. Consequently, the threaded portion further from the body of the patient during fitting is the distal end and this is to be provided with corresponding means for driving, not shown. Examples of possible drive means are a simple screw slot driver, a cross screw slot driver, an Inbus® or a Torx®.

The first, leading proximal threaded portion 3 is smaller in diameter than the second, driving distal threaded portion 4. The term smaller relates to all comparable diameter measurements. Thus, the external diameter of the proximal threaded portion is smaller than the external thread of the distal threaded portion 4. Likewise, the core diameter of the proximal threaded portion 3 is smaller than the core diameter of the distal threaded portion 4. In the dimensioning, for example, the core diameter of the distal threaded portion 4 may be approximately the same size as the external diameter of the proximal threaded portion 3. This approximately corresponds to the configuration as shown in FIG. 1. These size ratios may, however, also certainly vary, but, as already mentioned, comparable diameters of the proximal threaded portion 3 are always larger than the corresponding ones of the distal threaded portion 4.

The entire pin 2 is penetrated by a central bore or cannulation, as also emerges from WO 2009/115396. This cannulation is substantially used to guide a guide wire through. A guide wire of this type (determined here as a structural element whether a wire or a pin) is often called a Kirschner wire and is used for precise, linearly correct feeding of the reinforcement screw.

Two threads are cut onto the first proximal threaded portion 3. A first thread on the proximal threaded portion 3 forms the proximal, self-cutting fastening thread 7. The proximal fastening thread 7 has flanks, which enclose an acute angle, as described in the aforementioned WO 2009/115396. This acute angle is less than 45° and is preferably in the order of magnitude of between 10° and 20°. A proximal displacement thread 8 is located between two adjacent thread turns of the proximal fastening thread 7.

As this thread is designed to be self-cutting according to the invention and no pre-drilling takes place, a compression of the bone material already takes place when the reinforcement screw 1 is screwed in. A displacement thread is therefore optionally not imperative and its absence facilitates the screwing in operation.

The proximal displacement thread 8 has an external diameter which is substantially smaller than the external diameter of the proximal fastening thread 7. The proximal fastening thread 7 and the proximal displacement thread 8 both have the same core diameter. While the fastening screw 7 is used to anchor the screw in the proximally situated bone or bone part, the proximal displacement thread 8 has the object of displacing the spongy bone material present between the proximal fastening thread turns, so a bone material compression bringing about an improved anchoring of the reinforcement screw in the distal bone takes place as a result.

In an analogous manner, the second, distal threaded portion 4 has a distal fastening thread 5 and a distal displacement thread 6. The objects of the two distal threads precisely correspond to the objects which the corresponding proximal threads, as described above, have. The distal fastening thread 5 also has corresponding flanks here and the flanks of a thread turn also enclose an acute angle here. However, this will preferably be slightly less acute than the angle which the flanks of the proximal fastening thread 7 enclose. The corresponding angle is in the order of magnitude of between 20° and 40° here. In accordance with the larger diameter, the height of the distal displacement thread 6 is also correspondingly greater. The core diameter of the distal fastening thread 5 and of the distal displacement thread 6 is also the same size here. The external diameter of the displacement thread is also smaller in this portion than the external diameter of the fastening thread 5 that is present here. At maximum, the external diameter of the displacement thread 6 will preferably be selected approximately in accordance with the size of the flank diameter of the fastening thread 5. The external diameter of the displacement thread may also taper slightly from proximal to distal. This conicity is then present in the two threaded portions 3 and 4.

Owing to the relatively large thread pitch of the thread present here, it is difficult to insert a reinforcement screw of this type in a precisely aligning manner in the cavity provided after removal of the plug. Generally, initially only a small part of the proximal fastening thread 7 is namely located on the bone. Even if in this case a centering pin is guided through the cannulation, this is merely a certain orientation aid, but a real centering cannot be achieved in this manner. However, if the proximal fastening thread does not run precisely aligned with the centre axis in the two bones or bone parts, the two bones or bone parts are changed with respect to their relative position to one another. This is completely undesired and in order to avoid this, the reinforcement screw is provided with at least one, or preferably at least or precisely three, phase grinding faces 10 designed the same and distributed uniformly over the periphery. These phase grinding faces bring about a precise three-point contact during the introduction of the threaded portion 3 into the bone. This three-point contact guarantees a precise feed of the reinforcement screw.

The phase grinding faces 10 are not only used for the described three-point contact for introduction into the punched passage in the vertebral body S1 in a single level supply S1-L5 and in the lumbar vertebral body L5 in a double level supply (S1 to L4), but they also act as cutting phases in the lumbar vertebral body L5 that has not been pre-drilled (in single level supply) or the lumbar vertebral body L4 that has not been pre-drilled (in double level supply) for improved penetration of the corresponding cortical tissue plates of these vertebral bodies.

In principle, the fastening thread and the displacement thread in the respective same threaded portion have the same pitch. On the other hand, the pitch both of the fastening thread and the displacement thread of the proximal threaded portion is different from the two threads on the distal threaded portion 4. If a distraction is to be achieved, the thread pitch on the distal thread is selected to be greater than the pitch on the proximal end. However, if it is intended to achieve a contraction between the two bones or bone parts, the thread on the distal end will be provided with a smaller thread pitch than the thread on the proximal threaded portion. However, this is adequately known from the prior art.

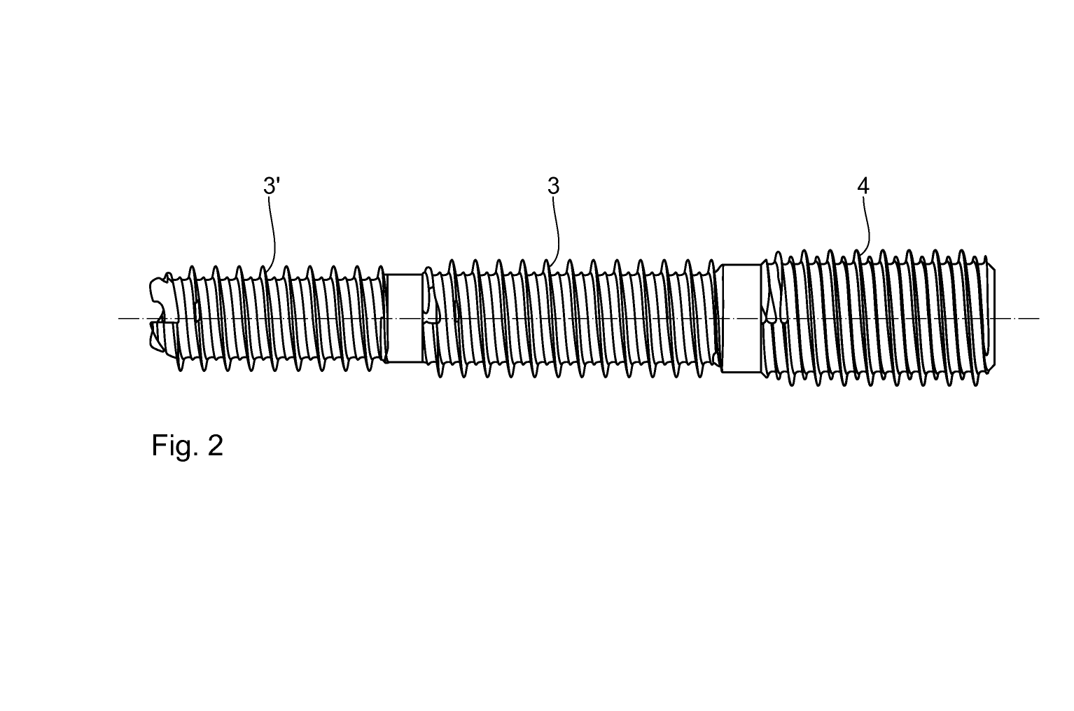

FIG. 2 shows a second embodiment of the reinforcement screw with two proximal threaded portions 3, 3′.

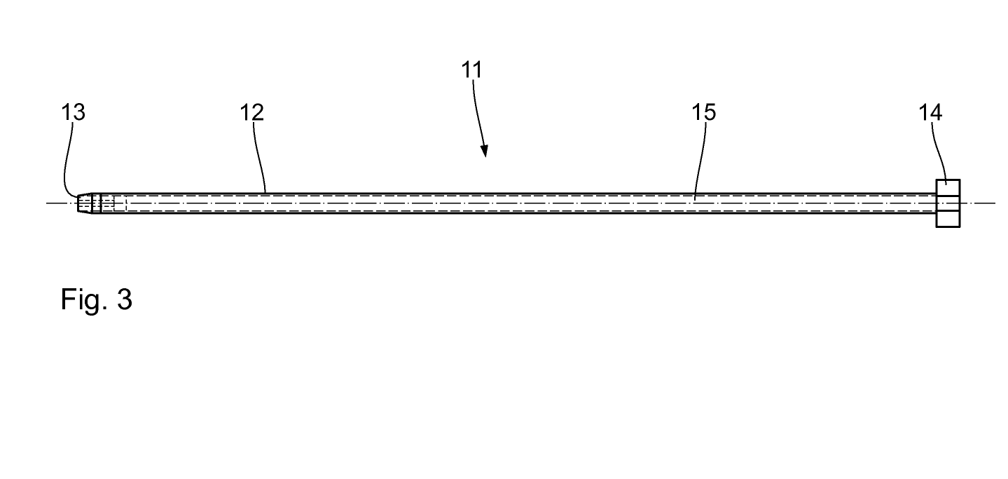

FIG. 3 shows a schematic view of a tool/bone plug removal punch for the secured removal of bone tissue 11 from vertebral bodies, comprising either a hollow cylinder 12 with any cross-section, provided with a toothed edge 13 at one end and a handle 14, or preferably a polygonal cross-section—preferably an octagon—with an inwardly cross-sectionally decreasing ground knife-sharp cutting edge 13 at one end and a handle 14. The polygonal configuration has the advantage of working with the smallest possible cross-sectional area, as the smallest diameter can be smaller than the core diameter of the reinforcement screw 1, the phase grinding faces 10 of which bring about an extension to the core diameter.

A plunger 15 for ejecting a bone plug is guided in the internal cross-section. The plunger 15 has a full cross-section, which is dimensioned such that it is guided at least approximately without play in the internal cross-section of the bone plug removal punch 12.

The toothed edge 13 of the hollow cylinder is provided with knife-like or chisel-like teeth in order to perforate the corticalis and thereby to easily cut through it. The spongy tissue can accordingly be passed through still more easily.

The bone plug removal punch 12 can, if necessary, also be provided with a displaceable and fixable adjustment element in order to be able to fix the penetration depth of the tool.

The tool 11 is firstly oriented and positioned in the usual manner and then driven by axial feeding through the corticalis of S1 toward the foot. It then penetrates the spongy tissue and finally the corticalis of S1 toward the head. The penetrated tissue remains secured as a “plug” in the cavity of the tool 11 and after its return, by means of the punch 15, is ejected unharmed and will be further used. If a reinforcement screw consisting of three portions is to be set, after the clearing out of the intervertebral disc tissue, in an analogous manner, a bone plug is removed from the vertebral body L5 using a thinner tool 11 (for example a diameter of 7 mm instead of 8 mm).

After the punching of the bone plug out of the vertebral body S1, the intervertebral disc intermediate space between 51 and L5 is cleared out in the conventional manner and filled up again with the removed bone tissue. For this purpose, the removed bone tissue is comminuted beforehand, for example in a bone mill and optionally mixed with bone replacement material.

The reinforcement screw 1 and tools are guided and/or set in the conventional manner by means of a tissue protection cover, not described in more detail, to protect the soft parts of the abdomen. The necessity for using the tissue protection cover arises, on the one hand, from the minimally invasive operation method and human anatomy, on the other hand, during access to the operation point.

The reinforcement screw 1 is screwed with the proximal threaded pin portion 3 leading into the vertebral body S1 or the cavity, the proximal threaded pin portion 3 gradually being screwed into the vertebral body L5. The adequately large diameter difference of the threads and the interruption of the thread between the threaded portions 3, 4 prevent a penetration of the threaded portion 4 into the vertebral body L5.

The distal threaded portion 4 can also optionally be provided with a self-cutting thread in order to only have to punch a bone plug with a very small cross-section from S1.

### LIST OF REFERENCE NUMERALS

1 reinforcement screw
2 pin
3 proximal threaded portion
4 distal threaded portion
5 distal fastening thread
6 distal displacement thread
7 proximal fastening thread
8 proximal displacement thread
9 cutting edge
10 phase grinding face
11 tool to remove bone tissue
12 hollow cylinder/bone plug removal punch
13 edge
14 handle
15 plunger

What is claimed is:

1. Instruments for reinforcing a human spinal column, comprising a tissue protection cover to keep open a position on the sacrum, at least one tool for removing intervertebral disc tissue and a reinforcement screw with at least a first, leading proximal threaded portion and a second, driving, distal threaded portion,
wherein a tool for the secured removal of bone tissue (11) from at least the last vertebral body S1 is furthermore provided and wherein the reinforcement screw (1) is head-free and, in the region of the proximal threaded portion (3), has one of the group of a self-cutting and self-centering fastening thread (7).

2. Instruments according to claim 1, wherein the reinforcement screw (1) comprises a pin (2) with a first, leading proximal threaded portion (3) and a second, driving distal threaded portion (4), wherein the diameter of the proximal threaded portion (3) is smaller than the diameter of the distal threaded portion (4) and the two threaded portions (3, 4) have different thread pitches, and wherein at least one of the two threaded portions (3, 4) is provided with a double thread, one thread being a fastening thread (5, 7) and the second thread being a displacement thread (6, 8), wherein at least one thread turn of the displacement thread runs between two adjacent thread turns of the fastening thread, in each case, and wherein the external diameter of the displacement thread is smaller than the external diameter of the fastening thread in the respective threaded portion both in the distal and in the proximal threaded portion (3, 4).

3. Instruments according to claim 1, wherein the tool to remove bone tissue (11) comprises a bone plug removal punch (12) with a hollow cylindrical cross-section.

4. Instruments according to claim 3, wherein one end of the bone plug removal punch (12) is provided with one of the group of a toothed edge and a sharp knife cutting edge (13) and the other end is provided with a handle (14).

5. Instruments according to claim 1, wherein the tool for removing bone tissue (11) comprises a bone plug removal punch (12) with a hollow polygonal cross-section.

6. Instrument according to claim 5, wherein one end of the bone plug removal punch (12) is provided with one of the group of a toothed edge and a sharp knife cutting edge (13) and the other end is provided with a handle (14).

7. Instruments according to claim 3, wherein an adjustable limiter of the penetration depth of the tool is provided on the bone plug removal punch (12).

8. Instruments according to claim 1, wherein the reinforcement screw (1) is provided with a cannulation extending through the entire length of the pin as one of the group of a central guide bore and for guiding the guide wire through.

9. Instruments according to claim 1, wherein in the reinforcement screw (1), a second portion with a self-cutting thread follows the first, distal portion with a self-cutting thread, the first portion being dimensioned such that this portion comes to rest approximately completely only in one of the group of the vertebral body L4 and L5 located furthest away on the head side.

10. Instruments according to claim 1, wherein the leading self-cutting threaded portion has a different pitch compared to the following threaded portion, so that a distraction is located between the foremost threaded portion and is achievable in the following vertebral body.

11. Instruments according to claim 1, wherein the reinforcement screw comprises three threaded portions and, in this case, has two distal threaded portions (3, 3′).

12. Instruments according to claim 1, wherein phase grinding faces (10) are provided as a 3-point contact on the reinforcement screw (1).

13. Instruments according to claim 1, wherein the phase grinding faces (10) are configured to penetrate the cortical base plates of one of the group of the lumbar vertebral bodies L5 and L4 to be correspondingly penetrated.

14. Instruments according to claim 3, wherein the bone plug removal punch (12) comprises a plunger (15) for ejecting the bone tissue removed in a secured manner.

15. Instruments according to claim 14, wherein the plunger (15) is configured in cross-section such that it is guided at least approximately without play in the cavity of the bone plug removal punch (12).

16. Instruments according to claim 15, wherein the internal cross-section of the bone plug removal punch (12) and the cross-section of the plunger (15) are the same apart from guide tolerances for the ejection without loss of material of the bone plug.
